(19) United States
(12) Patent Application Publication
(10) Pub. No.: US 2025/0009611 A1
(43) Pub. Date: Jan. 9, 2025

(54) NANO BUBBLE-CONTAINING COSMETIC COMPOSITION

(71) Applicants: SANSHO KAKEN K.K., Osaka (JP); Nano-Science Laboratory Corporation, Kyoto-shi (JP); H4 Co., Ltd., Osaka (JP)

(72) Inventors: Atsushi Ando, Osaka (JP); Katsuhito Hatanaka, Osaka (JP); Yuji Fujita, Kyoto-shi (JP); Tsuyoshi Kiriishi, Kyoto-shi (JP); Yosuke Takeshima, Osaka (JP)

(73) Assignees: SANSHO KAKEN K.K., Osaka (JP); Nano-Science Laboratory Corporation, Kyoto-shi (JP); H4 Co., Ltd., Osaka (JP)

(21) Appl. No.: 18/218,274

(22) Filed: Jul. 5, 2023

(51) Int. Cl. A61K 8/02; A61Q 5/00; A61Q 19/00; A61Q 19/10

(52) U.S. Cl. CPC A61K 8/0279; A61Q 5/00; A61Q 19/005; A61Q 19/007; A61Q 19/10; A61K 2800/33; A61K 2800/412; A61K 2800/413; A61K 2800/5922

(57) ABSTRACT

Provided is a cosmetic composition that conforms well to the skin and has an excellent feeling of use without causing stickiness due to residual oil even after application. A nano bubble-containing cosmetic composition according to the present invention relates to a nano bubble-containing cosmetic composition that comprises nano bubble-liquid and a cosmetic ingredient, wherein the nano bubble-liquid comprises oil and bubbles, the bubbles having a diameter of 5 nm to 500 nm.

### BACKGROUND OF THE INVENTION

### Field of the Invention

The present invention relates to a nano bubble-containing cosmetic composition. More specifically, it relates to a nano bubble-containing cosmetic composition comprising a nano bubble-liquid and a cosmetic ingredient.

### Description of the Related Art

Cosmetics are intended to be used on the body by rubbing, spraying, and other similar methods for cleaning, beautifying the human body, increasing attractiveness, changing appearance, or maintaining skin or hair healthy.

Cosmetics include for example, basic cosmetics intended for cleansing the skin such as soaps, toner, lotions, and creams; make-up cosmetics for adjusting skin tone and definition of the face by color tone such as foundations, rouges, powders, eyeshadows and nail polishes; and further hair care cosmetics, aromatic cosmetics, and medical cosmetics, etc. To date, various cosmetics are available on the market. In this specification, cosmetics also include quasi-drugs.

In recent years, modern people have become more interested in aesthetics, and a wide variety of generations, regardless of age or gender, are interested in keeping their skin beautiful. Specific methods for making the skin beautiful can include adjusting meal content, securing sufficient sleep, preventing ultraviolet rays and dryness, etc., and conditioning the skin, etc. In particular, a method for using cosmetics for conditioning the skin has been gathering public attention, since it is easy to obtain aesthetic effects.

Basic cosmetics for conditioning the skin can include for example, skin conditioning cosmetics such as skin oils, massage oils, essential oils, and cleansing oils, etc. Applying a skin conditioning cosmetic containing oil to the body enables suppression of moisture evaporation from the skin by the oil film and moisturization of the skin.

As stated above, a highly moisturizing sensation can be obtained by applying a skin conditioning cosmetic containing oil to the skin. However, depending on the type of oil used in the skin conditioning cosmetic or usage condition, some cosmetics provide a poor feeling of use, such as poor conformability to the skin and stickiness due to residual oil after application.

As a method of improving the feeling of use of such cosmetics, there is known a method in which a small amount of a surfactant having a low HLB is blended, whereby the surface tension of the oil can be reduced to improve the poor conformability to the skin and the stickiness due to residual oil.

A cosmetic composition comprising an oil-based ingredient to be applied to the skin includes for example, the one described in Japanese Unexamined Patent Application Publication No. 2001-213726. The document discloses an oil-based solid cleansing agent containing a solid oil with a melting point of 50 to 150° C., a liquid oil, and a nonionic surfactant with a HLB value of 5 to 15.

### SUMMARY OF THE INVENTION

### Problem to be Solved by the Invention

As stated above, Japanese Unexamined Patent Application Publication No. 2001-213726 discloses an oil-based solid cleansing agent containing a solid oil with a melting point of 50 to 150° C., a liquid oil, and a nonionic surfactant with a HLB value of 5 to 15. Compared to cosmetic compositions without a surfactant, such cosmetic composition allows the oil to conform well to the skin and suppresses skin from being oily and shiny due to residual oil.

On the other hand, there is the risk that a cosmetic composition comprising a surfactant can cause adverse effects, such as rough skin caused by the surfactant, to a user and this issue remains. In addition, if a user has an allergy to surfactants, the use of a cosmetic composition comprising a surfactant itself is accompanied by difficulties.

The present invention was made in view of the stated situation and provides a nano bubble-containing cosmetic composition comprising: a nano bubble-liquid; and a cosmetic ingredient, wherein the nano bubble-liquid comprises an oil and bubbles, the bubbles having a diameter of 5 nm to 500 nm.

According to the nano bubble-containing cosmetic composition of the present invention, nano bubble-containing cosmetic composition can be provided, wherein the bubbles having a diameter of 5 nm to 500 nm are dispersed in oil, and wherein the nano bubble-containing cosmetic composition conforms well to the skin, has no stickiness even after cosmetic application, and provides excellent feeling of use. In addition, since the nano bubble-containing cosmetic composition according to the present invention does not comprise a surfactant, the composition cannot cause adverse effects, such as rough skin to the user and can be used by even those who have allergies to surfactants.

### Means for Solving the Problem

The invention according to the first aspect relates to a nano bubble-containing cosmetic composition comprising: a nano bubble-liquid; and a cosmetic ingredient, wherein the nano bubble-liquid comprises an oil and bubbles, the bubbles having a diameter of 5 nm to 500 nm.

The invention according to the second aspect relates to the nano bubble-containing cosmetic composition of the first aspect, wherein the oil is one or more oils selected from the group consisting of at least squalane, mineral oil, 2-ethylhexyl palmitate, and olive oil.

The invention according to the third aspect relates to the nano bubble-containing cosmetic composition of the first aspect, wherein the bubbles are generated from one or more gases selected from the group consisting of hydrogen, carbon dioxide, air and oxygen.

The invention according to the fourth aspect relates to the nano bubble-containing cosmetic composition of the second aspect, wherein the bubbles are generated from one or more gases selected from the group consisting of hydrogen, carbon dioxide, air and oxygen.

The invention according to the fifth aspect relates to the nano bubble-containing cosmetic composition of the first aspect, wherein the nano bubble-containing cosmetic composition is a cleansing cosmetic composition, a skin conditioning cosmetic composition, a hair cosmetic composition or a bath cosmetic composition.

The invention according to the sixth aspect relates to the nano bubble-containing cosmetic composition of the second aspect, wherein the nano bubble-containing cosmetic composition is a cleansing cosmetic composition, a skin conditioning cosmetic composition, a hair cosmetic composition or a bath cosmetic composition.

The invention according to the seventh aspect relates to the nano bubble-containing cosmetic composition of the third aspect, wherein the nano bubble-containing cosmetic composition is a cleansing cosmetic composition, a skin conditioning cosmetic composition, a hair cosmetic composition or a bath cosmetic composition.

The invention according to the eighth aspect relates to the nano bubble-containing cosmetic composition of the fourth aspect, wherein the nano bubble-containing cosmetic composition is a cleansing cosmetic composition, a skin conditioning cosmetic composition, a hair cosmetic composition or a bath cosmetic composition.

The invention according to the nineth aspect relates to the nano bubble-containing cosmetic composition of the first aspect, wherein the nano bubble-containing cosmetic composition is a cosmetic composition containing the bubbles having a diameter of 80 to 140 nm to quickly conform to a skin.

The invention according to the tenth aspect relates to the nano bubble-containing cosmetic composition of the second aspect, wherein the nano bubble-containing cosmetic composition is a cosmetic composition containing the bubbles having a diameter of 80 to 140 nm to quickly conform to a skin.

The invention according to the eleventh aspect relates to the nano bubble-containing cosmetic composition of the third aspect, wherein the nano bubble-containing cosmetic composition is a cosmetic composition containing the bubbles having a diameter of 80 to 140 nm to quickly conform to a skin.

The invention according to the twelfth aspect relates to the nano bubble-containing cosmetic composition of the fourth aspect, wherein the nano bubble-containing cosmetic composition is a cosmetic composition containing the bubbles having a diameter of 80 to 140 nm to quickly conform to a skin.

The invention according to the thirteenth aspect relates to the nano bubble-containing cosmetic composition of the fifth aspect, wherein the nano bubble-containing cosmetic composition is a cosmetic composition containing the bubbles having a diameter of 80 to 140 nm to quickly conform to a skin.

The invention according to the fourteenth aspect relates to the nano bubble-containing cosmetic composition of the sixth aspect, wherein the nano bubble-containing cosmetic composition is a cosmetic composition containing the bubbles having a diameter of 80 to 140 nm to quickly conform to a skin.

The invention according to the fifteenth aspect relates to the nano bubble-containing cosmetic composition of the seventh aspect, wherein the nano bubble-containing cosmetic composition is a cosmetic composition containing the bubbles having a diameter of 80 to 140 nm to quickly conform to a skin.

The invention according to the sixteenth aspect relates to the nano bubble-containing cosmetic composition of the eighth aspect, wherein the nano bubble-containing cosmetic composition is a cosmetic composition containing the bubbles having a diameter of 80 to 140 nm to quickly conform to a skin.

### Effect of the Invention

According to the invention of the first aspect, since provided is a nano bubble-containing cosmetic composition comprising a nano bubble-liquid and a cosmetic ingredient, wherein the nano bubble-liquid comprises an oil and bubbles, the bubbles having a diameter of 5 nm to 500 nm, the invention can provide a nano bubble-containing cosmetic composition having an excellent feeling of use, wherein the oil quickly conforms to the skin and stickiness does not result from residual oil after cosmetic application. In addition, since the nano bubble-containing cosmetic composition does not comprise a surfactant, the composition cannot cause adverse effects, such as rough skin caused by a surfactant, to the user and can be used by even those who have allergies to surfactants. Further, since the invention uses the nano bubbles in which the bubbles have a diameter of 5 nm to 500 nm, it can provide a nano bubble-containing cosmetic composition, wherein the nano bubble-liquid can stably contain bubbles, and gas escaping cannot be caused for a long period of time.

According to the invention of the second aspect, since the oil is one or more oils selected from the group consisting of at least squalane, mineral oil, 2-ethylhexyl palmitate and olive oil, the invention can provide a nano bubble-containing cosmetic composition, wherein the nano bubble-liquid that uses such oil particularly easily conforms to the skin and the cosmetic composition has less stickiness even after cosmetic application.

According to the invention of the third aspect, since the bubbles are generated from one or more gases selected from the group consisting of hydrogen, carbon dioxide, air and oxygen, the invention can provide a warm-feeling to the skin by the nano bubble-liquid, comprised in the cosmetic composition, permeating the skin.

According to the invention of the fourth aspect, since the bubbles are generated from one or more gases selected from the group consisting of hydrogen, carbon dioxide, air and oxygen, the invention can provide a warm-feeling to the skin by the nano bubble-liquid, comprised in the cosmetic composition, permeating the skin.

According to the invention of the fifth aspect, since the nano bubble-containing cosmetic composition is a cleansing cosmetic composition, a skin conditioning cosmetic composition, a hair cosmetic composition or a bath cosmetic composition, the invention can provide a cleansing cosmetic composition, a skin conditioning cosmetic composition, a hair cosmetic composition or a bath cosmetic composition that can thoroughly remove the dirt on the skin, the scalp, etc. by quick conformation of oil to the skin, keep the skin and the scalp soft and healthy, and has an excellent feeling of use due to less stickiness caused by the oil.

According to the invention of the sixth aspect, since the nano bubble-containing cosmetic composition is a cleansing cosmetic composition, a skin conditioning cosmetic composition, a hair cosmetic composition or a bath cosmetic composition, the invention can provide a cleansing cosmetic composition, a skin conditioning cosmetic composition, a hair cosmetic composition or a bath cosmetic composition that can thoroughly remove the dirt on the skin, the scalp, etc. by quick conformation of oil to the skin, keep the skin and the scalp soft and healthy, and has an excellent feeling of use due to less stickiness caused by the oil.

According to the invention of the seventh aspect, since the nano bubble-containing cosmetic composition is a cleansing cosmetic composition, a skin conditioning cosmetic composition, a hair cosmetic composition or a bath cosmetic composition, the invention can provide a cleansing cosmetic composition, a skin conditioning cosmetic composition, a hair cosmetic composition or a bath cosmetic composition that can thoroughly remove the dirt on the skin, the scalp, etc. by quick conformation of oil to the skin, keep the skin and the scalp soft and healthy, and has an excellent feeling of use due to less stickiness caused by the oil.

According to the invention of the eighth aspect, since the nano bubble-containing cosmetic composition is a cleansing cosmetic composition, a skin conditioning cosmetic composition, a hair cosmetic composition or a bath cosmetic composition, the invention can provide a cleansing cosmetic composition, a skin conditioning cosmetic composition, a hair cosmetic composition or a bath cosmetic composition that can thoroughly remove the dirt on skin, the scalp, etc. by quick conformation of oil to the skin, keep the skin and the scalp soft and healthy, and has an excellent feeling of use due to less stickiness caused by the oil.

According to the invention of the nineth aspect, since the nano bubble-containing cosmetic composition is a cosmetic composition containing the bubbles having a diameter of 80 to 140 nm to quickly conform to a skin, the invention can provide a nano bubble-containing cosmetic composition that can suppress the evaporation of moisture from the skin by the quick permeance of oil to the skin and has high moisture retention.

According to the invention of the tenth aspect, since the nano bubble-containing cosmetic composition is a cosmetic composition containing the bubbles having a diameter of 80 to 140 nm to quickly conform to a skin, the invention can provide a nano bubble-containing cosmetic composition that can suppress the evaporation of moisture from the skin by the quick permeance of oil to the skin and has high moisture retention.

According to the invention of the eleventh aspect, since the nano bubble-containing cosmetic composition is a cosmetic composition containing the bubbles having a diameter of 80 to 140 nm to quickly conform to a skin, the invention can provide a nano bubble-containing cosmetic composition that can suppress the evaporation of moisture from the skin by the quick permeance of oil to the skin and has high moisture retention.

According to the invention of the twelfth aspect, since the nano bubble-containing cosmetic composition is a cosmetic composition containing the bubbles having a diameter of 80 to 140 nm to quickly conform to a skin, the invention can provide a nano bubble-containing cosmetic composition that can suppress the evaporation of moisture from the skin by the quick permeance of oil to the skin and has high moisture retention.

According to the invention of the thirteenth aspect, since the nano bubble-containing cosmetic composition is a cosmetic composition containing the bubbles having a diameter of 80 to 140 nm to quickly conform to a skin, the invention can provide a nano bubble-containing cosmetic composition that can suppress the evaporation of moisture from the skin by the quick permeance of oil to the skin and has high moisture retention.

According to the invention of the fourteenth aspect, since the nano bubble-containing cosmetic composition is a cosmetic composition containing the bubbles having a diameter of 80 to 140 nm to quickly conform to a skin, the invention can provide a nano bubble-containing cosmetic composition that can suppress the evaporation of moisture from the skin by the quick permeance of oil to the skin and has high moisture retention.

According to the invention of the fifteenth aspect, since the nano bubble-containing cosmetic composition is a cosmetic composition containing the bubbles having a diameter of 80 to 140 nm to quickly conform to a skin, the invention can provide a nano bubble-containing cosmetic composition that can suppress the evaporation of moisture from the skin by the quick permeance of oil to the skin and has high moisture retention.

According to the invention of the sixteenth aspect, since the nano bubble-containing cosmetic composition is a cosmetic composition containing the bubbles having a diameter of 80 to 140 nm to quickly conform to a skin, the invention can provide a nano bubble-containing cosmetic composition that can suppress the evaporation of moisture from the skin by the quick permeance of oil to the skin and has high moisture retention.

### BRIEF DESCRIPTION OF DRAWINGS

FIG. 1 is a schematic configuration diagram of a nano bubble generator required for the present invention.

### DETAILED DESCRIPTION OF THE INVENTION

Suitable embodiments of the nano bubble-containing cosmetic composition (hereinafter, also simply referred to as cosmetic composition) according to the examples of the present invention are described below.

The present cosmetic composition comprises a nano bubble-liquid and a cosmetic ingredient. A nano bubble-liquid refers to a liquid having nano bubbles dispersed in oil.

Nano bubble is a general term for a fine bubble contained in a liquid (water or oil), referring to a bubble having a diameter of less than 1 μm. Further, a bubble, as used herein, refers to a gas bubble formed in a liquid.

Fine bubble refers to a bubble having a diameter of 100 μm or less, and is classified into a micro bubble and nano bubble (ultrafine bubble). The bubble of the micro bubble has a diameter from 1 μm to 100 μm, while the bubble of the nano bubble has a diameter of less than 1 μm as stated above.

Although any nano bubble-liquid can be used as the nano bubble-liquid in the present cosmetic composition can be used if it has a diameter of less than 1 μm, from the view point of quality maintenance of the present cosmetic composition, it is desirable to use a device that can produce a nano bubble-liquid having a similar concentration, uniformity and stability of nano bubbles, and a very small amount of nano particles to be mixed, when compared to the nano bubble generator used for the present invention (Nano-Science Laboratory Corporation, Model Name “Lab-nano-GS-2A-PD51-F-2106”).

In addition, as the nano bubble-liquid in the present cosmetic composition, desirably the liquid with bubbles having a diameter from 5 nm to 500 nm, more desirably bubbles having a diameter from 50 nm to 500 nm is used. When the bubbles have a diameter greater than 500 nm, the gas can escape from the solution due to the large bubble size, and thus, it becomes difficult to preserve the nano bubble-liquid for a long time. Further, an average diameter of the bubbles is desirably 80 to 140 nm. When the bubbles have an average diameter greater than 140 nm, the gas can escape from the solution due to the large bubble size, and thus, it becomes difficult to preserve the nano bubble-liquid for a long time.

Since the present cosmetic composition contains the nano bubble-liquid, the bubbles comprised in the cosmetic can be preserved for a long period of time when compared to a micro bubble-containing cosmetic composition. Thus, the present cosmetic composition can be suitably used as a cosmetic that can be preserved for a long time. In addition, the inventors focused on the fact that nano bubbles have better permeability than micro bubbles, and found that the use of the nano bubble-liquid in the present cosmetic composition can enables quick conformation of oil to the skin and elimination of stickiness due to residual oil. Therefore, the nano bubble-liquid needs to be used in the present cosmetic composition. Furthermore, the nano bubble-liquid has an effect of promoting blood flow, and the present cosmetic composition can be more suitably used as a cosmetic that can also provide a warm-feeling to the skin.

As the oil in the present cosmetic composition, for example, squalane, mineral oil, olive oil, jojoba oil, sweet almond oil, grapeseed oil, apricot kernel oil, macadamia nut oil, sunflower oil, camelia oil, sesame oil, argan oil, raspberry seed oil, coconut oil, walnut oil, kukui nut oil, passion flower oil, peach kernel oil, hazelnut oil, hemp seed oil, avocado oil, aloe vera oil, evening primrose oil, wheat germ oil, castor oil, orange seed oil, hippophae oil (sea buckthorn oil), flaxseed oil (linseed oil), rosehip oil, prune seed oil, andiroba oil, Inca Inchi oil, cashew nut oil, camelina oil, kiwi seed oil, cucumber seed oil, guava seed oil, cranberry seed oil, grapefruit seed oil, rice oil, bran oil, rice bran oil, arnica oil, carrot oil, comfrey oil, etc. can be used. In addition, ester oil can be used as the oil in the present cosmetic composition, for example, 2-ethylhexyl palmitate, isopropyl myristate, isopropyl palmitate, tri (caprylic/capric acid) glyceryl, 2-ethylhexyl isononanoate, isononyl isononanoate, etc. Further, higher alcohol can be used as the oil in the present cosmetic composition, for example, oleyl alcohol, etc. In addition, silicone oil can be used as the oil in the present cosmetic composition, for example, octamethyltrisiloxane, decamethyltetrasiloxane, methyl polysiloxane, methyl phenyl polysiloxane (phenyl methicone), decamethylcyclopentasiloxane volatile silicone, octamethylcyclotetrasiloxane volatile silicone, etc. Moreover, it is most preferable to use at least one or more of squalane, mineral oil, 2-ethylhexyl palmitate or olive oil as the oil in the present cosmetic composition. The nano bubble-liquid that uses such oil particularly conforms to the skin easily, and has less stickiness even after cosmetic application.

Although any gas obvious to a person skilled in the art can be used in the present cosmetic composition, it is desirable to use hydrogen, carbon dioxide, air, or oxygen etc., or a combination thereof may be used. These gases easily permeate the skin, and can provide a warm-feeling to the skin.

As the cosmetic ingredient used in the present cosmetic composition, for example, a moisturizer (softener, emollient), astringent (antiperspirant), cooling agent, whitening agent, UV absorbent or other agents can be used. However, since the cosmetic ingredient is not limited to these examples, any ingredient obvious to a person skilled in the art can be used.

As a moisturizer, for example, diglycerin, dipropylene glycol, mixed solution of glycosyl trehalose and hydrogenated starch degradation products, 1,2-pentanediol, 1,2-hexanediol, propanediol, maltitol, sorbitol, glucose, xylitol, 1,2-octanediol, ethylhexylglycerin, sodium acetylated hyaluronate, hydrolyzed hyaluronic acid, hydroxypropyltrimonium hyaluronate, etc. can be used.

As an astringent, for example, citric acid, lactic acid, aluminium sulfate, lemon water, hamamelis, etc. can be used.

As a cooling agent, for example, menthol, ethyl alcohol, camphor, or eucalyptus oil, etc. can be used.

As a whitening agent, for example, arbutin, tranexamic acid, vitamin C derivative, or placenta, etc. can be used.

As a UV absorber, for example, PABA, t-butyl methoxydibenzoylmethane, oxybenzone-1, oxybenzone-2, oxybenzone-3, oxybenzone-4, oxybenzone-5, oxybenzone-6, oxybenzone-9, polysilicone-15, ethylhexyl methoxycinnamate, or diethylamino hydroxybenzoyl hexyl benzoate, etc. can be used.

As other agents, for example, an acne agent, dandruff/itching agent, hircismus inhibitor, sterilizer/disinfectant (dipotassium glycyrrhizate, etc.), a cosmetic such as a hair tonic, or a quasi-drug such as a hair restoration product, etc. can be used.

The present cosmetic composition may further comprise an additive, plant extract or thickener.

As an additive, for example, a preservative, antioxidant, or metal sequestering agent (metal ion element sequestering agent), etc. can be used.

As a preservative, for example, silver oxide, paraoxybenzoic acid esters, sorbic acid, sodium dehydroacetate, quaternary ammonium salt (benzalkonium chloride, benzethonium chloride, etc.), chloroxidine, pentylene glycol, or phenoxyethanol, etc. can be used.

As an antioxidant, for example, tocopherol (vitamin E), ascorbic acid, dibutylhydroxytoluene (BHT), or butylated hydroxyanisole (BHA), etc. can be used.

As a metal sequestering agent, for example, chelating agents (sodium edetate or ethylenediaminetetraacetate (EDTA)), etc. can be used.

As a plant extract, dokudami extract, Phellodendron Bark extract, melilot extract, dead nettle extract, licorice extract, peony extract, soapwort extract, loofah extract, kina extract, saxifraga extract, sophora flavescens extract, fennel extract, primula extract, rose extract, dioecy extract, lemon extract, lithospermum extract, aloe extract, ginger root extract, eucalyptus extract, horsetail extract, sage extract, thyme extract, tea extract, seaweed extract, cucumber extract, clove extract, raspberry extract, melissa extract, carrot extract, horse chestnut extract, peach leaf extract, mulberry extract, cornflower extract, hamamelis extract, ougon extract, apple extract, yuzu extract, artichoke extract, grapefruit extract, etc. can be used.

As a thickener, a macromolecular component, etc. can be used. For example, a natural water-soluble polymer, semisynthetic water-soluble polymer, synthetic water-soluble polymer, or inorganic water-soluble polymer, etc. can be used as a macromolecular component.

As a natural water-soluble polymer, for example, a plant-based polymer such as gum arabic, tragacanth gum, galactan, guar gum, carrageenan, pectin, agar, quince seed (marmelo), alge colloid, starch (rice, corn, potato, wheat), glycyrrhizic acid, etc., a microbial polymer such as xanthan gum, dextran, succinoglycan, pullulan, etc., or an animal polymer such as casein, albumin, gelatin, etc. can be used.

As a semisynthetic water-soluble polymer, for example, a starch polymer such as carboxymethyl starch methyl, hydroxypropyl starch, etc., a cellulose polymer such as methyl cellulose, ethyl cellulose, methyl hydroxypropyl cellulose, hydroxyethyl cellulose, hydroxypropyl cellulose, sodium carboxymethyl cellulose (CMC), crystal cellulose, cellulose powder, etc., and an alginate polymer such as sodium alginate, propylene glycol esters of alginate, etc. can be used.

As a synthetic water-soluble polymer, for example, polyvinyl alcohol, polyvinyl methyl ether, polyvinylpyrrolidone, polyethylene glycol, sodium polyacrylate, poly ethyl acrylate, polyacrylamide, etc. can be used.

As an inorganic water-soluble polymer, for example, bentonite, AlMg silicate (veegum), Laponite, hectorite, silicic anhydride, etc. can be used.

The present cosmetic composition can comprise a vitamin extract or animal extract (such as collagen) to provide an excellent moisturizing effect to the cosmetic composition.

The present cosmetic composition can be suitably used, for example, as a cleansing cosmetic composition such as a cleansing oil. Users can thoroughly remove oil stains and/or applied cosmetics on their skin by quickly conforming the present cosmetic composition to their skin. In addition, since the present cosmetic composition has less stickiness caused by the oil, the composition allows for comfortable cleansing and has an excellent feeling of use. Furthermore, when hydrogen, oxygen, carbon dioxide, etc. is used as the nano bubbles in the present cosmetic composition, the nano bubbles can provide a warm-feeling to the skin, and such cosmetic composition thus can be used as a more suitable cleansing oil than a cleansing cosmetic composition with a surfactant.

The present cosmetic composition can be suitably used, for example, as a skin conditioning cosmetic composition such as a skin oil or a massage oil. Users can comfortably carry out a massage without damaging their skin by quickly conforming the present cosmetic composition to their skin. In addition, since the present cosmetic composition has less stickiness caused by the oil, the composition has an excellent feeling of use. Furthermore, when hydrogen, oxygen, carbon dioxide, etc. is used as the nano bubbles in the present cosmetic composition, the nano bubbles can provide a warm-feeling to the skin. Thus, such cosmetic composition can be used as a more suitable skin conditioning cosmetic composition than a skin conditioning cosmetic composition with a surfactant.

The present cosmetic composition can be suitably used, for example, as a hair cosmetic composition such as a hair oil or a hair tonic. Users can make their scalp healthy and their hair soft by quickly conforming the present cosmetic composition to their scalp or hair. Since the present cosmetic composition has less stickiness caused by the oil, the composition has an excellent feeling of use. Furthermore, when hydrogen, oxygen, carbon dioxide, etc. is used as the nano bubbles in the present cosmetic composition, the nano bubbles can provide a warm-feeling to the scalp, and such cosmetic composition thus can be used as a more suitable hair cosmetic composition than a hair cosmetic composition with a surfactant.

The present cosmetic composition can be suitably used, for example, as a bath cosmetic composition. For example, in the use of the present cosmetic composition during taking a bath, users can soften and protect the entire skin by conforming the present cosmetic composition to their skin over the entire body. In addition, when hydrogen, oxygen, carbon dioxide, etc. is used as the nano bubbles in the present cosmetic composition, the nano bubbles can provide a warm-feeling to the skin, and such cosmetic composition thus can have an excellent feeling of use, and can be used as a more suitable bath cosmetic composition than a bath cosmetic composition with a surfactant.

Suitable embodiments of a production method of the cosmetic composition according to the examples of the present invention are described below.

The present cosmetic composition can be produced by the method comprising steps of (a) supplying a gas to an oil to obtain a nano bubble-liquid with bubbles having a diameter of 5 nm to 500 nm, and (b) blending the oil with a cosmetic ingredient, wherein steps (a) and (b) are performed in any sequence or simultaneously.

An oil similar to the oil used in the present cosmetic composition can be used.

The nano bubble-liquid is prepared by supplying gas to the oil.

The nano bubble-liquid is produced using a nano bubble generator (Nano-Science Laboratory Corporation, Model Name “Lab-nano-GS-2A-PD51-F-2106”). Any nano bubble generator can be used in the production of the nano bubble-liquid if a nano bubble-liquid is produced with a similar concentration, uniformity and stability of nano bubbles, and a very small amount of nano particles to be mixed.

The nano bubble concentration is 1×10⁶ (count/ml) or more, preferably 1×10⁷ (count/ml) or more.

Nano bubbles with high uniformity are nano bubbles with bubble diameters of 5 to 500 nm, 50 to 300 nm, 80 to 200 nm or 80 to 140 nm, and have a small variation in bubble diameter.

A nano bubble-liquid with significantly high stability is a nano bubble-liquid that maintains a nano bubble concentration of 1×10⁷ (count/ml) for at least 3, 5, 6, 12 or 15 months after the oil is contained in the nano bubbles.

The nano bubbles according to the present invention are characterized by significantly high uniformity and stability compared to nano bubbles that are generated by conventional swirling flow, static mixer, ejector, venturi, pressurized micropore or pressurized dissolution-type nano bubble generators.

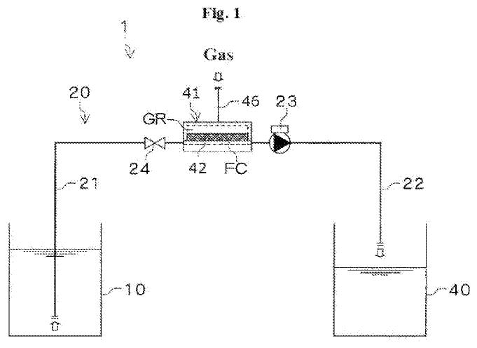

The nano bubble generator (Nano-Science Laboratory Corporation, Model Name “Lab-nano-GS-2A-PD51-F-2106”) used for the present invention is described in detail using FIG. 1.

FIG. 1 depicts a schematic configuration of the nano bubble generator (Nano-Science Laboratory Corporation, Model Name “Lab-nano-GS-2A-PD51-F-2106”) used in the present invention. As indicated in the figure, the nano bubble generator 1 used for the present invention comprises a liquid storage tank 10 that stores a liquid before a gas is released; a liquid sending unit 20 (relative moving means) that sucks up and sends out the liquid stored in the liquid storage tank 10; a gas release unit 41 that releases nano bubbles into the liquid while the liquid is sent by the liquid sending unit 20; and the liquid storage tank 40 that stores the liquid comprising a gas released by the gas release unit 41.

The liquid sending unit 20 comprises a liquid sending pipe 21 that forms a liquid flow path; a liquid sending pump 23 of a variable flow type provided in a liquid sending pipe portion 22; and a valve 24 provided in the liquid sending pipe portion 21 for adjusting the degree of negative pressure of the gas release unit 41, so that the liquid stored in the liquid storage tank 10 is sent through the gas release unit 41 to the liquid storage tank 40.

The gas release unit 41 comprises a gas release component 42, and a liquid flow path FC is formed with the liquid in contact with the lower surface (gas release surface) of the gas release component 42. The gas to be released is supplied into the gas supply chamber GR through the gas supply tube 46, and the gas is provided to liquid as nano bubbles through the gas release component 42.

Although any gas obvious to a person skilled in the art can be used, it is desirable to use hydrogen, carbon dioxide, air, or oxygen etc.

The liquid stored in the liquid storage tank 10 is sent from the liquid sending pipe 21 of the liquid sending unit 20 to the gas release unit 41, then the liquid is sent to the liquid sending pipe 22 of the liquid sending unit 20 through the flow path FC, and then the liquid comes out from the liquid sending pipe to the liquid storage tank 40.

The liquid used in the present invention is primarily oil, and preferably the viscosity of the oil is 1000 mPa-s or less.

The gas release component 42 consists of a vented porous material formed of porous ceramics such as porous alumina, porous glass, etc., and has a lower surface with a plurality of gas release holes having a pore diameter (modal diameter) of 1.5 [μm] or less. Specifically, there are six types of pore diameter (modal diameter) of the gas release holes, i.e., 1.5 μm, 1 μm, 0.8 μm, 0.4 μm, 0.05 μm and 0.005 μm.

In the nano bubble generator 1 configured as above, the driving of the liquid sending pump 23 after introduction of the liquid into the liquid storage tank 10 allows the liquid in the liquid storage tank 10 to be sent to the liquid storage tank 40 through the flow path of the gas release unit 41, while negative pressure is generated in the flow path FC of the gas release unit 41 that is located on the suction side of the liquid sending pump 23.

This negative pressure allows the air to be sucked from the gas release holes provided on the lower surface of the gas release component 42 into the liquid passing through the flow path FC. The pumping flow rate is adjusted so that the liquid flow velocity in the flow path FC of the gas release unit 41 is 0.3 [m/sec] or higher.

The gas, which is sucked from the gas release holes of the gas release component 42 into the liquid passing through the flow path FC by the movement of the liquid, is dispersed into micro bubbles of 1.5 μm or less by the liquid flow flowing through the flow path FC, and these micro bubbles are slowly shrunken to generate nano-order micro bubbles, thereby the liquid containing the nano bubbles are stored in the liquid storage tank.

The liquid sending pump is a fluid path independent pump and has a structure that does not generate dust upon pump operation, and desirably has a large lift, and preferably a pump having a lift of 5 m or more may be used.

In addition, the conventional swirling flow, static mixer, ejector, venturi, pressurized micropore, or pressurized dissolution-type nano bubble generators also generate micro bubbles simultaneously to cause cavitation in the device. This cavitation causes a small amount of erosion in the device, resulting in contamination of nano particles which are impurities. Since this contamination of the nano particles is measured incorrectly as nano bubbles when the nano bubble concentration is measured, the contamination of nano particles is undesirable.

On the other hand, since the nano bubble generator used for the present invention generates only nano bubbles, and the liquid sending pump is a fluid path independent pump and has a structure that does not generate dust upon pump operation, there is very little contamination of nano particles which are impurities.

A cosmetic ingredient that is similar to the cosmetic ingredient used in the present cosmetic composition can be used.

As steps (a) and (b) are performed in any sequence or simultaneously, any of the following patterns may be taken. (1) The present cosmetic composition is obtained by supplying the gas to the oil to obtain a nano bubble-liquid, which is blended with the cosmetic ingredient (performing in the sequence of step (a), step (b)). (2) The present cosmetic composition is obtained by blending the oil with the cosmetic ingredient, and then supplying the gas to obtain a nano bubble-liquid (performing in the sequence of step (b), step (a)). (3) The present cosmetic composition is obtained by blending the oil with the cosmetic ingredient while supplying the gas (performing step (a) and step (b) simultaneously). The present cosmetic composition can be produced in any of the patterns described above.

In addition, it is not necessary to supply and/or dissolve the gas and/or cosmetic ingredient in the total oil volume. For example, the present cosmetic composition may be obtained by dividing the total oil volume into two, supplying a gas to the first oil to prepare the nano bubble-liquid, dissolving the cosmetic ingredients in the second oil to prepare the oil, and then blending the nano bubble-liquid with the oil in which the cosmetic ingredient is dissolved.

A gas similar to the gas used in the present cosmetic composition can be used.

The present cosmetic composition can be suitably used, for example, as a cleansing cosmetic composition such as a cleansing oil. Users can thoroughly remove oil stains and/or applied cosmetics on their skin by quickly conforming the present cosmetic composition to their skin. In addition, since the present cosmetic composition has less stickiness caused by the oil, the composition allows for comfortable cleansing and has an excellent feeling of use. Furthermore, when hydrogen, oxygen, carbon dioxide, etc. is used as the nano bubbles in the present cosmetic composition, the nano bubbles can provide a warm-feeling to the skin, and such cosmetic composition thus can be used as a more suitable cleansing cosmetic composition than a cleansing cosmetic composition with a surfactant.

The present cosmetic composition can be suitably used, for example, as a skin conditioning cosmetic composition such as a skin oil or a massage oil. Users can comfortably carry out a massage without damaging their skin by quickly conforming the present cosmetic composition to their skin. In addition, since the present cosmetic composition has less stickiness caused by the oil, the composition has an excellent feeling of use. Furthermore, when hydrogen, oxygen, carbon dioxide, etc. is used as the nano bubbles in the present cosmetic composition, the nano bubbles can provide a warm-feeling to the skin. Thus, such cosmetic composition can be used as a more suitable skin conditioning cosmetic composition than a skin conditioning cosmetic composition with a surfactant.

The present cosmetic composition can be suitably used, for example, as a hair cosmetic composition such as a hair oil or a hair tonic. Users can make their scalp healthy and their hair soft by quickly conforming the present cosmetic composition to their scalp or hair. In addition, the present cosmetic composition has less stickiness caused by the oil, the composition has an excellent feeling of use. Furthermore, when hydrogen, oxygen, carbon dioxide, etc. is used as the nano bubbles in the present cosmetic composition, the nano bubbles can provide a warm-feeling to the scalp, and such cosmetic composition thus can be used as a more suitable hair cosmetic composition than a hair cosmetic composition with a surfactant.

The present cosmetic composition can be suitably used, for example, as a bath cosmetic composition. For example, in the use of the present cosmetic composition that is a bath cosmetic composition during taking a bath, users can soften and protect the entire skin by conforming the present cosmetic composition to their skin over the entire body. In addition, when hydrogen, oxygen, carbon dioxide, etc. is used as the nano bubbles in the present cosmetic composition, the nano bubbles can provide a warm-feeling to the skin, and such cosmetic composition thus can have an excellent feeling of use and can be used as a more suitable bath cosmetic composition than a bath cosmetic composition with a surfactant.

### EXAMPLES

Examples of a nano bubble-containing cosmetic composition according to the present invention are shown below to further clarify the effects of the present invention. However, the present invention is not limited to the following examples in any way.

### Example 1

(Preparation of a Cosmetic Composition Containing Nano Bubbles Generated from Hydrogen Gas)

2-ethylhexyl palmitate was aerated with nano bubbles generated from hydrogen gas, which were generated by a nano bubble generator (Nano-Science Laboratory Corporation, Model Name “Lab-nano-GS-2A-PD51-F-2106”), to prepare a nano bubble-liquid. Tocopherol was then mixed with the prepared nano bubble-liquid as a cosmetic ingredient to prepare the nano bubble-containing cosmetic composition of Example 1. The blending ratio of the nano bubble-containing cosmetic composition of Example 1 was shown in Table 1.

### Example 2

(Preparation of a Cosmetic Composition Containing Nano Bubbles Generated from Oxygen Gas)

2-ethylhexyl palmitate was aerated with nano bubbles generated from oxygen gas, which were generated by a nano bubble generator (Nano-Science Laboratory Corporation, Model Name “Lab-nano-GS-2A-PD51-F-2106”), to prepare a nano bubble-liquid. Tocopherol was then mixed with the prepared nano bubble-liquid as a cosmetic ingredient to prepare the nano bubble-containing cosmetic composition of Example 2. The blending ratio of the nano bubble-containing cosmetic composition of Example 2 was shown in Table 1.

### Example 3

(Preparation of a Cosmetic Composition Containing Nano Bubbles Generated from Carbon Dioxide Gas)

2-ethylhexyl palmitate was aerated with nano bubbles generated from carbon dioxide gas, which were generated by a nano bubble generator (Nano-Science Laboratory Corporation, Model Name “Lab-nano-GS-2A-PD51-F-2106”), to prepare a nano bubble-liquid. Tocopherol was then mixed with the prepared nano bubble-liquid as a cosmetic ingredient to prepare the nano bubble-containing cosmetic composition of Example 3. The blending ratio of the nano bubble-containing cosmetic composition of Example 3 was shown in Table 1.

### Example 4

(Preparation of a Cosmetic Composition Containing Nano Bubbles Generated from Hydrogen Gas)

Squalane was aerated with nano bubbles generated from hydrogen gas, which were generated by a nano bubble generator (Nano-Science Laboratory Corporation, Model Name “Lab-nano-GS-2A-PD51-F-2106”), to prepare a nano bubble-liquid. Tocopherol was then mixed with the prepared nano bubble-liquid as a cosmetic ingredient to prepare the nano bubble-containing cosmetic composition of Example 4. The blending ratio of the nano bubble-containing cosmetic composition of Example 4 was shown in Table 1.

### Comparative Example 1

### (Preparation of an Oil-Containing Cosmetic Composition)

2-ethylhexyl palmitate and tocopherol as a cosmetic ingredient were mixed to prepare an oil-containing cosmetic composition. The blending ratio of the oil-containing cosmetic composition of Comparative Example 1 was shown in Table 1.

### Comparative Example 2

### (Preparation of an Oil-Containing Cosmetic Base Comprising a Surfactant)

Instead of tocopherol used in Comparative Example 1, 2-ethylhexyl palmitate was blended with sorbitan monoisostearate, which is a surfactant, to prepare an oil-containing cosmetic base. The blending ratio of the oil-containing cosmetic base of Comparative Example 2 was shown in Table 1.

### Test Methods

After applying 1 mL of each sample of the nano bubble-containing cosmetic compositions of Examples 1-4, the oil-containing cosmetic composition of Comparative Example 1 and the oil-containing cosmetic base of Comparative Example 2 to the forearm of a person, the forearm was massaged with the fingertips, and the degree of conformity to the skin and the feeling of use were assessed by sensory assessment. The efficacy evaluation test results are shown in Table 1. The effects of the ingredients and samples were assessed in Table 1 as follows: ∘ indicates that the sample quickly conforms to the skin after application, has no stickiness due to residual oil, and has an excellent feeling of use; Δ indicates that the sample conforms to the skin after application, however, has stickiness due to residual oil; and x indicates that the oil remains on the skin after application and the skin is oily and shiny due to residual oil. In addition, in the row for nano bubbles in Table 1, ∘ shows the presence of nano bubbles, x shows the absence of nano bubbles, and the type of gas used is shown in parentheses. Furthermore, tocopherol was blended into the samples of Examples 1-4 and Comparative Example 1 at 0.1% as a cosmetic ingredient.

TABLE 1
Efficacy Evaluation Test Results 1

| | Sample | | | | | |
|---|---|---|---|---|---|---|
| Ingredient | Example | Example | Example | Example | Comparative | Comparative |
| Name | 1 | 2 | 3 | 4 | Example 1 | Example 2 |
| 2-ethylhexyl | 99.9 | 99.9 | 99.9 | | 99.9 | 99.8 |
| palmitate | | | | | | |
| Squalane | | | | 99.9 | | |
| Tocopherol | 0.1 | 0.1 | 0.1 | 0.1 | 0.1 | |
| Sorbitan | | | | | | 0.2 |
| monoisostearate | | | | | | |
| Nano bubbles | ◯ | ◯ | ◯ | ◯ | X | X |
| | (Hydrogen) | (Oxygen) | (Carbon | (Hydrogen) | | |
| | | | dioxide) | | | |
| Effect | ◯ | ◯ | ◯ | ◯ | X | Δ |

### Example 5

(Preparation of a Cosmetic Composition Containing Nano Bubbles Generated from Hydrogen Gas)

Olive oil was aerated with nano bubbles generated from hydrogen gas, which were generated by a nano bubble generator (Nano-Science Laboratory Corporation, Model Name “Lab-nano-GS-2A-PD51-F-2106”), to prepare a nano bubble-liquid. Tocopherol was then mixed with the prepared nano bubble-liquid as a cosmetic ingredient to prepare the nano bubble-containing cosmetic composition of Example 5. The blending ratio of the nano bubble-containing cosmetic composition of Example 5 was shown in Table 2.

### Example 6

(Preparation of a Cosmetic Composition Containing Nano Bubbles Generated from Oxygen Gas)

Olive oil was aerated with nano bubbles generated from oxygen gas, which were generated by a nano bubble generator (Nano-Science Laboratory Corporation, Model Name “Lab-nano-GS-2A-PD51-F-2106”), to prepare a nano bubble-liquid. Tocopherol was then mixed with the prepared nano bubble-liquid as a cosmetic ingredient to prepare the nano bubble-containing cosmetic composition of Example 6. The blending ratio of the nano bubble-containing cosmetic composition of Example 6 was shown in Table 2.

### Example 7

(Preparation of a Cosmetic Composition Containing Nano Bubbles Generated from Carbon Dioxide Gas)

Olive oil was aerated with nano bubbles generated from carbon dioxide gas, which were generated by a nano bubble generator (Nano-Science Laboratory Corporation, Model Name “Lab-nano-GS-2A-PD51-F-2106”), to prepare a nano bubble-liquid. Tocopherol was then mixed with the prepared nano bubble-liquid as a cosmetic ingredient to prepare the nano bubble-containing cosmetic composition of Example 7. The blending ratio of the nano bubble-containing cosmetic composition of Example 7 was shown in Table 2.

### Comparative Example 3

### (Preparation of an Oil-Containing Cosmetic Composition)

Olive oil and tocopherol as a cosmetic ingredient were mixed to prepare an oil-containing cosmetic composition. The blending ratio of the oil-containing cosmetic composition of Comparative Example 3 was shown in Table 2.

### Comparative Example 4

### (Preparation of an Oil-Containing Cosmetic Base Comprising a Surfactant)

Instead of tocopherol used in Comparative Example 3, olive oil was blended with sorbitan monoisostearate, which is a surfactant, to prepare an oil-containing cosmetic base. The blending ratio of the oil-containing cosmetic base of Comparative Example 4 was shown in Table 2.

### Test Methods

After applying 1 mL of each sample of the nano bubble-containing cosmetic compositions of Examples 5-7, the oil-containing cosmetic composition of Comparative Example 3 and the oil-containing cosmetic base of Comparative Example 4 to the forearm of a person, the forearm was massaged with the fingertips, and the degree of conformity to the skin and the feeling of use were assessed by sensory assessment. The efficacy evaluation test results are shown in Table 2. The effects of the ingredients and samples were assessed in Table 2 as follows: ∘ indicates that the sample quickly conforms to the skin after application, has no stickiness due to residual oil, and has an excellent feeling of use; Δ indicates that the sample conforms to the skin after application, however, has stickiness due to residual oil; and x indicates that the oil remains on the skin after application and the skin is oily and shiny due to residual oil. In addition, in the row for nano bubbles in Table 2, ∘ shows the presence of nano bubbles, x shows the absence of nano bubbles, and the type of gas used is shown in parentheses. Furthermore, tocopherol was blended into the samples of Examples 5-7 and Comparative Example 3 at 0.1% as a cosmetic ingredient.

TABLE 2
Efficacy Evaluation Test Results 2

| | Sample | | | | |
|---|---|---|---|---|---|
| Ingredient | Example | Example | Example | Comparative | Comparative |
| Name | 5 | 6 | 7 | Example 3 | Example 4 |
| Olive Oil | 99.9 | 99.9 | 99.9 | 99.9 | 99.8 |
| Tocopherol | 0.1 | 0.1 | 0.1 | 0.1 | |
| Sorbitan | | | | | 0.2 |
| mono- | | | | | |
| isostearate | | | | | |
| Nano | ○ | ○ | ○ | × | × |
| bubbles | (Hydro- | (Oxygen) | (Carbon | | |
| | gen) | | dioxide) | | |
| Effect | ○ | ○ | ○ | × | Δ |

### Example 8

(Preparation of a Cosmetic Composition Containing Nano Bubbles Generated from Hydrogen Gas)

Mineral oil was aerated with nano bubbles generated from hydrogen gas, which were generated by a nano bubble generator (Nano-Science Laboratory Corporation, Model Name “Lab-nano-GS-2A-PD51-F-2106”), to prepare a nano bubble-liquid. Tocopherol was then mixed with the prepared nano bubble-liquid as a cosmetic ingredient to prepare the nano bubble-containing cosmetic composition of Example 8. The blending ratio of the nano bubble-containing cosmetic composition of Example 8 was shown in Table 3.

### Example 9

(Preparation of a cosmetic composition containing nano bubbles generated from oxygen gas)

Mineral oil was aerated with nano bubbles generated from oxygen gas, which were generated by a nano bubble generator (Nano-Science Laboratory Corporation, Model Name “Lab-nano-GS-2A-PD51-F-2106”), to prepare a nano bubble-liquid. Tocopherol was then mixed with the prepared nano bubble-liquid as a cosmetic ingredient to prepare the nano bubble-containing cosmetic composition of Example 9. The blending ratio of the nano bubble-containing cosmetic composition of Example 9 was shown in Table 3.

### Example 10

(Preparation of a Cosmetic Composition Containing Nano Bubbles Generated from Carbon Dioxide Gas)

Mineral oil was aerated with nano bubbles generated from carbon dioxide gas, which were generated by a nano bubble generator (Nano-Science Laboratory Corporation, Model Name “Lab-nano-GS-2A-PD51-F-2106”), to prepare a nano bubble-liquid. Tocopherol was then mixed with the prepared nano bubble-liquid as a cosmetic ingredient to prepare the nano bubble-containing cosmetic composition of Example 10. The blending ratio of the nano bubble-containing cosmetic composition of Example 10 was shown in Table 3.

### Comparative Example 5

### (Preparation of an Oil-Containing Cosmetic Composition)

Mineral oil and tocopherol as a cosmetic ingredient were mixed to prepare an oil-containing cosmetic composition. The blending ratio of the oil-containing cosmetic composition of Comparative Example 5 was shown in Table 3.

### Comparative Example 6

### (Preparation of an Oil-Containing Cosmetic Base Comprising a Surfactant)

Instead of tocopherol used in Comparative Example 5, mineral oil was blended with sorbitan monoisostearate, which is a surfactant, to prepare an oil-containing cosmetic base. The blending ratio of the oil-containing cosmetic base of Comparative Example 6 was shown in Table 3.

### Test Methods

After applying 1 mL of each sample of the nano bubble-containing cosmetic compositions of Examples 8-10, the oil-containing cosmetic composition of Comparative Example 5 and the oil-containing cosmetic base of Comparative Example 6 to the forearm of a person, the forearm was massaged with the fingertips, and the degree of conformity to the skin and the feeling of use were assessed by sensory assessment. The efficacy evaluation test results are shown in Table 3. The effects of the ingredients and samples were assessed in Table 3 as follows: ∘ indicates that the sample quickly conforms to the skin after application, has no stickiness due to residual oil, and has an excellent feeling of use; Δ indicates that the sample conforms to the skin after application, however, has stickiness due to residual oil; and x indicates that oil remains on the skin after application and the skin is oily and shiny due to residual oil. In addition, in the row for nano bubbles in Table 3, ∘ shows the presence of nano bubbles, x shows the absence of nano bubbles, and the type of gas used is shown in parentheses. Furthermore, tocopherol was blended into the samples of Examples 8-10 and Comparative Example 5 at 0.1% as a cosmetic ingredient.

TABLE 3
Efficacy Evaluation Test Results 3

| | Sample | | | | |
|---|---|---|---|---|---|
| Ingredient | Example | Example | Example | Comparative | Comparative |
| Name | 8 | 9 | 10 | Example 5 | Example 6 |
| Mineral oil | 99.9 | 99.9 | 99.9 | 99.9 | 99.8 |
| Tocopherol | 0.1 | 0.1 | 0.1 | 0.1 | |
| Sorbitan | | | | | 0.2 |
| mono- | | | | | |
| isostearate | | | | | |
| Nano | ○ | ○ | ○ | × | × |
| bubbles | (Hydro- | (Oxygen) | (Carbon | | |
| | gen) | | dioxide) | | |
| Effect | ○ | ○ | ○ | × | Δ |

### (Measurement of the Nano Bubble Stability in a Nano Bubble-Containing Cosmetic Composition)

Stability of the nano bubbles used in the present invention was compared and measured under the following conditions.

The nano bubble-containing oil used in Examples 11-22 was made using a nano bubble generator (Nano-Science Laboratory Corporation, Model Name “Lab-nano-GS-2A-PD51-F-2106”). Specifically, the nano bubble-containing oil was made by generating nano bubbles of each gas (hydrogen, carbon dioxide, oxygen and air) in 1 L of each oil (2-ethylhexyl palmitate, olive oil and mineral oil). The nano bubble-containing oil was then allowed to stand for 30 minutes after preparation, the prepared nano bubble-containing oil was then mixed with tocopherol as a cosmetic ingredient to prepare a nano bubble-containing cosmetic composition.

The nano bubble-containing oil used in Comparative Examples 7-9 was made by a high-speed rotation of a propeller. Specifically, the nano bubble-containing oil was made by filling a sealed 1 L container with each oil (2-ethylhexyl palmitate, olive oil and mineral oil) in the absence of air and rotating the propeller attached to the completely sealed rotating axis at a high-speed rotation (10,000 rpm) for 1 hour. The nano bubble-containing oil was then allowed to stand for 30 minutes after preparation, the prepared nano bubble-containing oil was then mixed with tocopherol as a cosmetic ingredient to prepare the nano bubble-containing cosmetic composition.

The nano bubble-containing oil used in Comparative Examples 10-12 was made using a nano bubble generator (means for generating nano bubbles by cavitation of the micro bubbles that are generated by the micro bubble generator nozzle). Specifically, the nano bubble-containing oil was made by generating micro bubbles in 1 L of each oil (2-ethylhexyl palmitate, olive oil and mineral oil) using the micro bubble generator nozzle and irradiating 40 kHz ultrasonic waves with an oscillator to cause cavitation. After the micro bubbles were extinguished by allowing the nano bubble-containing oil to stand for 60 minutes after preparation, and the prepared nano bubble-containing oil was then mixed with tocopherol as a cosmetic ingredient to prepare the nano bubble-containing cosmetic composition.

The oils and gases included in the Examples and Comparative Examples respectively are shown in Table 4.

TABLE 4

| Examples | Gases | Oils | Nano Bubble Generators |
|---|---|---|---|
| Example 11 | Hydrogen | 2-ethylhexyl | Nano bubble |
| Example 12 | Oxygen | palmitate | generator (Nano- |
| Example 13 | Carbon | | Science Laboratory |
| | dioxide | | Corporation, Model |
| Example 14 | Air | | Name “Lab-nano- |
| Example 15 | Hydrogen | Olive oil | GS-2A-PD51-F- |
| Example 16 | Oxygen | | 2106”) |
| Example 17 | Carbon | | |
| | dioxide | | |
| Example 18 | Air | | |
| Example 19 | Hydrogen | Mineral oil | |
| Example 20 | Oxygen | | |
| Example 21 | Carbon | | |
| | dioxide | | |
| Example 22 | Air | | |
| Comparative | Dissolved air | 2-ethylhexyl | High-speed rotation |
| Example 7 | in vaporized | palmitate | of a propeller |
| | oil | | |
| Comparative | Dissolved air | Olive oil | |
| Example 8 | in vaporized | | |
| | oil | | |
| Comparative | Dissolved air | Mineral oil | |
| Example 9 | in vaporized | | |
| | oil | | |
| Comparative | Air | 2-ethylhexyl | |
| Example 10 | | palmitate | |
| Comparative | Air | Olive oil | Nano bubbles |
| Example 11 | | | generation by |
| Comparative | Air | Mineral oil | cavitation of micro |
| Example 12 | | | bubbles |

### (Measurement of Nano Bubbles in a Nano Bubble-Containing Cosmetic Composition)

Measurement of nano bubble concentration in the nano bubble-containing cosmetic composition was carried out using a concentration measuring device manufactured by Nano-Science Laboratory Corporation.

Nano bubble concentration (count/ml) was measured for 50 cc of each nano bubble-containing cosmetic compositions containing nano bubbles of each gas (hydrogen, carbon dioxide, oxygen and air) in each oil (2-ethylhexyl palmitate, olive oil and mineral oil) for each elapsed time.

The storage conditions for each nano bubble-containing cosmetic composition were set to be under room temperature and normal pressure. Room temperature here refers to 1 to 30° C., and normal pressure refers to under atmospheric pressure.

The measurement results are shown below in Table 5.

### Table 5

TABLE 5
Stability Test Results (count/ml)

| Elapsed | Example | Example | Example | Example | Comparative | Comparative |
|---|---|---|---|---|---|---|
| Time | 11 | 12 | 13 | 14 | Example 7 | Example 10 |
| Immediately | 3.14 × 10⁸ | 5.27 × 10⁸ | 1.27 × 10⁸ | 8.25 × 10⁸ | 7.58 × 10⁷ | 7.18 × 10⁷ |
| after | | | | | | |
| generation | | | | | | |
| One day | 2.78 × 10⁸ | 4.58 × 10⁸ | 1.25 × 10⁸ | 8.22 × 10⁸ | 5.28 × 10⁶ | 6.68 × 10⁶ |
| One week | 2.55 × 10⁸ | 4.49 × 10⁸ | 1.14 × 10⁸ | 8.22 × 10⁸ | 3.11 × 10³ | 5.11 × 10⁴ |
| One month | 2.45 × 10⁸ | 4.44 × 10⁸ | 8.78 × 10⁷ | 7.18 × 10⁸ | Unmeasurable | Unmeasurable |
| Three months | 1.48 × 10⁸ | 3.20 × 10⁸ | 6.92 × 10⁷ | 5.55 × 10⁸ | Unmeasurable | Unmeasurable |
| Six months | 9.84 × 10⁷ | 8.68 × 10⁷ | 5.87 × 10⁷ | 4.28 × 10⁸ | Unmeasurable | Unmeasurable |
| Elapsed | Example | Example | Example | Example | Comparative | Comparative |
| Time | 15 | 16 | 17 | 18 | Example 8 | Example 11 |
| Immediately | 5.89 × 10⁸ | 7.89 × 10⁸ | 3.33 × 10⁸ | 1.21 × 10⁹ | 8.58 × 10⁶ | 7.38 × 10⁶ |
| after | | | | | | |
| generation | | | | | | |
| One day | 3.68 × 10⁸ | 5.58 × 10⁸ | 1.25 × 10⁸ | 9.92 × 10⁸ | 1.28 × 10⁶ | 6.98 × 10⁶ |
| One week | 3.55 × 10⁸ | 5.31 × 10⁸ | 1.14 × 10⁸ | 9.82 × 10⁸ | 6.51 × 10⁴ | 8.51 × 10⁵ |
| One month | 3.12 × 10⁸ | 4.94 × 10⁸ | 9.75 × 10⁷ | 7.19 × 10⁸ | Unmeasurable | Unmeasurable |
| Three months | 2.48 × 10⁸ | 4.15 × 10⁸ | 8.96 × 10⁷ | 6.45 × 10⁸ | Unmeasurable | Unmeasurable |
| Six months | 8.25 × 10⁷ | 2.58 × 10⁸ | 8.85 × 10⁷ | 5.23 × 10⁸ | Unmeasurable | Unmeasurable |
| Elapsed | Example | Example | Example | Example | Comparative | Comparative |
| Time | 19 | 20 | 21 | 22 | Example 9 | Example 12 |
| Immediately | 4.14 × 10⁸ | 8.27 × 10⁸ | 5.27 × 10⁸ | 8.64 × 10⁸ | 1.58 × 10⁷ | 3.58 × 10⁷ |
| after | | | | | | |
| generation | | | | | | |
| One day | 3.23 × 10⁸ | 4.58 × 10⁸ | 2.35 × 10⁸ | 8.35 × 10⁸ | 3.27 × 10⁶ | 8.27 × 10⁶ |
| One week | 2.12 × 10⁸ | 4.49 × 10⁸ | 2.81 × 10⁸ | 7.99 × 10⁸ | 8.67 × 10³ | 5.67 × 10⁴ |
| One month | 2.32 × 10⁸ | 4.44 × 10⁸ | 9.91 × 10⁷ | 7.58 × 10⁸ | Unmeasurable | Unmeasurable |
| Three months | 1.56 × 10⁸ | 3.28 × 10⁸ | 8.94 × 10⁷ | 6.55 × 10⁸ | Unmeasurable | Unmeasurable |
| Six months | 1.25 × 10⁸ | 2.65 × 10⁸ | 6.75 × 10⁷ | 5.12 × 10⁸ | Unmeasurable | Unmeasurable |

As shown in Table 5, the nano bubble-containing cosmetic compositions manufactured using a high-speed rotation of a propeller, which is one of the conventional methods to generate nano bubbles (Comparative Examples 7-9), have rapidly decreased nano bubble concentration one week after production, and become unmeasurable one month after production. Similarly, the nano bubble-containing cosmetic compositions manufactured by cavitation of micro bubbles, which is one of the conventional methods to generate nano bubbles (Comparative Examples 10-12), also have rapidly decreased nanobubble concentration one week after production, and become unmeasurable one month after production.

On the other hand, the nano bubble-containing cosmetic compositions manufactured by the nano bubble generator used for the present invention (Nano-Science Laboratory Corporation, Model Name “Lab-nano-GS-2A-PD51-F-2106”) show almost no change in nano bubble concentration even after an elapse of six months, maintaining the concentration of 1.0×10⁷ (count/ml) or more.

Accordingly, the nano bubbles generated by the conventional nano bubble generator and the nano bubbles generated by the nano bubble generator used for the present invention (Nano-Science Laboratory Corporation, Model Name “Lab-nano-GS-2A-PD51-F-2106”) have different properties when compared to nano bubbles that are generated by the conventional nano bubble generator and have more stability than conventional nano bubbles, allowing the nano bubbles to be used in cosmetics and other applications that require long-term stability.

### DISCUSSION

The efficacy evaluation test results in Table 1, which use 2-ethylhexyl palmitate as an oil, are discussed.

The oil-containing cosmetic composition of Comparative Example 1 showed to provide poor feeling of use because the oil did not conform to the skin even after application and the oil remained sticky.

The oil-containing cosmetic base of Comparative Example 2 showed to have the oil slowly conform to the skin after application since the base contains sorbitan monoisostearate as a surfactant, and showed that the permeability issue is improved by adding a surfactant to oil. However, the base remained to have stickiness due to residual oil and showed that an improvement needs to be made to the feeling of use.

The nano bubble-containing cosmetic composition in Examples 1-3 showed to quickly conform to the skin after cosmetic application and have no stickiness due to the residual oil even after application, and thus, showed to have improved the feeling of use. In addition, the nano bubble-containing cosmetic compositions of Examples 1-3 showed to have the oil quickly conform to the skin and have an excellent feeling of use without a surfactant, and thus, cannot cause adverse effects, such as rough skin caused by a surfactant, to a user of the oil and can be suitably used as a usable cosmetic composition even by those who have allergies to surfactants. Further, in Examples 1-3, three types of gases, i.e., hydrogen, oxygen and carbon dioxide, were considered as a gas in the nano bubbles, and all of these gases showed to have improved the conformity to the skin and the feeling of use, which indicates that the cosmetic compositions with these gases can be suitably used.

The nano bubble-containing cosmetic composition of Example 4, in which the oil was changed from 2-ethylhexyl palmitate to squalane, quickly conforms to the skin after cosmetic application and has no stickiness due to residual oil even after application, which indicates that the nano bubble-containing cosmetic composition with squalane as an oil can also be suitably used.

In the efficacy evaluation test results in Table 2, which use olive oil as an oil, a similar trend was observed as in the efficacy evaluation test results in Table 1. In other words, the nano bubble-containing cosmetic compositions of Examples 5-7 showed to quickly conform to the skin after cosmetic application and have no stickiness due to residual oil even after application, indicating that the feeling of use has improved.

In the efficacy evaluation test results in Table 3, which use mineral oil as an oil, a similar trend was observed as in the efficacy evaluation test results in Table 1 and Table 2. In other words, the nano bubble-containing cosmetic compositions of Examples 8-10 showed to quickly conform to the skin after cosmetic application and have no stickiness due to residual oil even after application, indicating that the feeling of use has improved.

Accordingly, it is shown that the nano bubble-containing cosmetic composition of the present invention can be suitably used as a nano bubble-containing cosmetic composition that quickly conforms to the skin after cosmetic application and has no stickiness due to residual oil even if various oils are used.

### INDUSTRIAL APPLICABILITY

According to the nano bubble-containing cosmetic composition of the present invention, since it is a nano bubble-containing cosmetic composition comprising a nano bubble-liquid and a cosmetic ingredient, wherein the nano bubble-liquid comprises an oil and bubbles, the bubbles having a diameter of 5 nm to 500 nm, the invention can provide a nano bubble-containing cosmetic composition having an excellent feeling of use, wherein the oil quickly conforms to the skin and stickiness does not result from residual oil after cosmetic application. In addition, since the nano bubble-containing cosmetic composition does not comprise a surfactant, the composition cannot cause adverse effects, such as rough skin caused by a surfactant, to the user and can be used by even those who have allergies to surfactants. Further, since the invention uses the nano bubbles in which the bubbles have a diameter of 5 nm to 500 nm, it can provide a nano bubble-containing cosmetic composition, wherein the nano bubble-liquid can stably contain bubbles, and gas escaping cannot be caused for a long period of time.

Accordingly, the nano bubble-containing cosmetic composition of the present invention can be suitably and widely used as a cosmetic composition that conforms well to the skin and has an excellent feeling of use without causing stickiness due to residual oil even after application.

1. A nano bubble-containing cosmetic composition comprising: a nano bubble-liquid; and a cosmetic ingredient, wherein the nano bubble-liquid comprises an oil and bubbles, the bubbles having a diameter of 5 nm to 500 nm.

2. The nano bubble-containing cosmetic composition of claim 1, wherein the oil is one or more oils selected from the group consisting of at least squalene, mineral oil, 2-ethylhexyl palmitate and olive oil.

3. The nano bubble-containing cosmetic composition of claim 1, wherein the bubbles are generated from one or more gases selected from the group consisting of hydrogen, carbon dioxide, air and oxygen.

4. The nano bubble-containing cosmetic composition of claim 2, wherein the bubbles are generated from one or more gases selected from the group consisting of hydrogen, carbon dioxide, air and oxygen.

5. The nano bubble-containing cosmetic composition of claim 1, wherein the nano bubble-containing cosmetic composition is a cleansing cosmetic composition, a skin conditioning cosmetic composition, a hair cosmetic composition or a bath cosmetic composition.

6. The nano bubble-containing cosmetic composition of claim 2, wherein the nano bubble-containing cosmetic composition is a cleansing cosmetic composition, a skin conditioning cosmetic composition, a hair cosmetic composition or a bath cosmetic composition.

7. The nano bubble-containing cosmetic composition of claim 3, wherein the nano bubble-containing cosmetic composition is a cleansing cosmetic composition, a skin conditioning cosmetic composition, a hair cosmetic composition or a bath cosmetic composition.

8. The nano bubble-containing cosmetic composition of claim 4, wherein the nano bubble-containing cosmetic composition is a cleansing cosmetic composition, a skin conditioning cosmetic composition, a hair cosmetic composition or a bath cosmetic composition.

9. The nano bubble-containing cosmetic composition of claim 1, wherein the nano bubble-containing cosmetic composition is a cosmetic composition containing the bubbles having a diameter of 80 to 140 nm to quickly conform to a skin.

10. The nano bubble-containing cosmetic composition of claim 2, wherein the nano bubble-containing cosmetic composition is a cosmetic composition containing the bubbles having a diameter of 80 to 140 nm to quickly conform to a skin.

11. The nano bubble-containing cosmetic composition of claim 3, wherein the nano bubble-containing cosmetic composition is a cosmetic composition containing the bubbles having a diameter of 80 to 140 nm to quickly conform to a skin.

12. The nano bubble-containing cosmetic composition of claim 4, wherein the nano bubble-containing cosmetic composition is a cosmetic composition containing the bubbles having a diameter of 80 to 140 nm to quickly conform to a skin.

13. The nano bubble-containing cosmetic composition of claim 5, wherein the nano bubble-containing cosmetic composition is a cosmetic composition containing the bubbles having a diameter of 80 to 140 nm to quickly conform to a skin.

14. The nano bubble-containing cosmetic composition of claim 6, wherein the nano bubble-containing cosmetic composition is a cosmetic composition containing the bubbles having a diameter of 80 to 140 nm to quickly conform to a skin.

15. The nano bubble-containing cosmetic composition of claim 7, wherein the nano bubble-containing cosmetic composition is a cosmetic composition containing the bubbles having a diameter of 80 to 140 nm to quickly conform to a skin.

16. The nano bubble-containing cosmetic composition of claim 8, wherein the nano bubble-containing cosmetic composition is a cosmetic composition containing the bubbles having a diameter of 80 to 140 nm to quickly conform to a skin.
